(19) United States
(12) Patent Application Publication
(10) Pub. No.: US 2025/0331938 A1
(43) Pub. Date: Oct. 30, 2025

(54) MECHANICAL TELEOPERATED DEVICE COMPRISING AN INTERCHANGEABLE DISTAL INSTRUMENT

(71) Applicant: Distalmotion SA, Epalinges (CH)

(72) Inventor: Ricardo Daniel Rita BEIRA, Lausanne (CH)

(21) Appl. No.: 19/208,428

(22) Filed: May 14, 2025

Related U.S. Application Data
(62) Division of application No. 18/311,177, filed on May 2, 2023, now Pat. No. 12,329,481.
Application No. 18/311,177 is a continuation of application No. 16/389,854, filed on Apr. 19, 2019, now abandoned.
Application No. 16/389,854 is a continuation of application No. 15/116,509, filed on Aug. 3, 2016, now Pat. No. 10,265,129.

(30) Foreign Application Priority Data
Feb. 3, 2014 (CH) CH00138/14
Mar. 12, 2014 (EP) 14159025.7
Mar. 18, 2014 (CH) CH00408/14

(51) Int. Cl. A61B 34/35; A61B 17/00; A61B 34/00; A61B 34/30; A61B 34/37; A61B 90/50; B25J 3/02

(52) U.S. Cl. CPC A61B 34/35; A61B 34/30; A61B 34/37; A61B 34/71; B25J 3/02; A61B 2017/00477; A61B 2090/506

(57) ABSTRACT

Disclosed is a mechanical teleoperated device comprising i) a handle (30) placed in a proximal part of the device, having a plurality of handle links interconnected by respective handle joints, ii) an end effector (31) placed in a distal part of the device, having a plurality of end-effector links interconnected by respective end-effector joints, iii) a transmission system arranged to kinematically connect the end effector to the handle such that movements of the end effector correspond to movements of the handle, comprising a) at least one arrangement of a plurality of rotatable elements (41a-d) coaxially mounted together about an axis and arranged to rotate independently from each other, b) first transmission means comprising driving elements (62a-c) which are arranged to actuate in rotation of the plurality of rotatable elements (41a-d) based upon movements of the handle, c) second transmission means comprising driven elements (63a-c) which are arranged to be driven by rotation of the plurality of rotatable elements resulting in movement of the end effector links, wherein the transmission system is configured for removably coupling together the first and second transmission means wherein each driven element is removably coupled to one corresponding rotatable element.

### CROSS-REFERENCE TO RELATED APPLICATIONS

This application is a divisional of U.S. patent application Ser. No. 18/311,177, filed May 2, 2023, which is a continuation of U.S. patent application Ser. No. 16/389,854, filed Apr. 19, 2019, which is a continuation of U.S. patent application Ser. No. 15/116,509, filed Aug. 3, 2016, now U.S. Pat. No. 10,265,129, which is the U.S. National Phase under 35 U.S.C. § 371 of International Patent Application No. PCT/EP2015/051473, filed Jan. 26, 2015, which claims priority to Switzerland Patent Application No. CH00408/14, filed Mar. 18, 2014, EPO patent application Ser. No. 14159025.7, filed Mar. 12, 2014, and Switzerland Patent Application No. CH00138/14, filed Feb. 3, 2014, the entire contents of each of which are incorporated herein by reference.

### FIELD OF THE INVENTION

The present invention relates to the field of remotely actuated mechanical systems. In particular embodiments, the invention relates to a mechanical teleoperated device for use primarily in surgical procedures and comprising an interchangeable distal instrument. Certain embodiments have particular applicability in minimally invasive surgical procedures. The present invention is also suitable for any remotely actuated application requiring dexterous manipulation with high stiffness and precision along with force feedback. Examples of such applications include assembly manipulation, manipulation in narrow places, manipulation in dangerous or difficult environments, and manipulation in contaminated or clean-room environments.

### BACKGROUND OF THE INVENTION

Numerous environments and applications call for remote actuation with mechanically teleoperated devices. These applications include fine manipulation in assembly tasks, manipulation in narrow places, manipulation in dangerous or contaminated environments, manipulation in clean-room or sterile environments and manipulation in surgical environments, whether open field or minimally invasive. While these applications vary along parameters such as precise tolerances and typical end user, each demands many of the same features from a mechanically teleoperated system, such as the ability to carry out dexterous manipulation with high stiffness and precision along with force feedback.

Surgical applications are now discussed in more detail as a representative example of an application for a mechanically teleoperated device system where known devices exist but significant shortcomings are evident in the current state of the art.

Open surgery is still the standard technique for most surgical procedures. It has been used by the medical community for several decades and consists of performing the surgical tasks by making a long incision in the abdomen or other area of the body, through which traditional surgical tools are inserted. However, due to the long incision, this approach is extremely invasive for patients, resulting in substantial blood loss during surgery and, typically, long and painful recovery periods in a hospital setting.

In order to reduce the invasiveness of open surgery, laparoscopy, a minimally invasive technique, was developed. Instead of a single long incision, several small incisions are made in the patient through which long and thin surgical instruments and endoscopic cameras are inserted. Because of the minimally invasive nature of the procedure, this technique reduces blood loss and pain and shortens hospital stays. When performed by experienced surgeons, this technique can attain clinical outcomes similar to open surgery. However, despite the above-mentioned advantages, laparoscopy requires extremely advanced surgical skill to manipulate the rigid and long instrumentation. The entry incision acts as a point of rotation, decreasing the freedom for positioning and orientating the instruments inside the patient. The movements of the surgeon's hand about this incision are inverted and scaled-up relative to the instrument tip (“fulcrum effect”), which reduces dexterity and sensitivity and magnifies the tremors of the surgeon hands. In addition, the long and straight instruments force the surgeon to work in an uncomfortable posture for hands, arms and body, which can be tremendously tiring during several hours of an operation. Therefore, due to these drawbacks of laparoscopic instrumentation, these minimally invasive techniques are mainly limited to use in simple surgeries, while only a small minority of surgeons is able to use them in complex procedures.

To overcome these limitations, surgical robotic systems were developed to provide an easier-to-use approach to complex minimally invasive surgeries. By means of a computerized robotic interface, these systems enable the performance of remote laparoscopy where the surgeon sits at a console manipulating two master manipulators to perform the operation through several small incisions. Like laparoscopy, the robotic approach is also minimally invasive, bringing the above-mentioned advantages over open surgery in terms of pain, blood loss, and recovery time. In addition, it also offers better ergonomy for the surgeon compared to open and laparoscopic techniques. However, although being technically easier, robotic surgery brings several negative aspects. A major disadvantage of these systems relates to the extremely high complexity of the existing robotic devices, which comprise complex mechatronic systems, leading to huge costs of acquisition and maintenance, which are not affordable for the majority of surgical departments worldwide. Another drawback of these systems comes from the fact that current surgical robots are large, competing for precious space within the operating room environment and significantly increasing preparation time. Access to the patient is thus impaired, which, together with a general lack of force-feedback, raises safety concerns.

WO9743942, WO9825666 and US2010011900 disclose a robotic tele-operated surgical instrument, designed to replicate surgeons' hand movements inside the patient's body. By means of a computerized, robotic interface, it enables the performance of remote laparoscopy, wherein the surgeon sits at a console manipulating two joysticks to perform the operation through several small incisions. However, this system does not have autonomy or artificial intelligence, being essentially a sophisticated tool fully controlled by the surgeon. The control commands are transmitted between the robotic master and robotic slave by a complex computer-controlled mechatronic system, which is extremely costly to produce and maintain and difficult to use for the hospital staff.

WO2012049623 describes a mechanical manipulator for surgical instruments with a master-slave configuration and including remote actuation of a distal end effector. However, the system described therein does not provide for an interchangeable instrument.

WO2013014621 describes a mechanical teleoperated device for remote manipulation which comprises master-slave configuration including a slave unit driven by a kinematically equivalent master unit such that each part of the slave unit mimics the movement of each corresponding part of the master unit. Although the mechanical transmission system is well adapted to the device, cables passing through an axial joint are twisted when the device is operating, which can force the cables to rub against each other. This increases wear on the cables over time and increases friction in the overall mechanical transmission.

Accordingly, an aim of the present invention is to provide a mechanical teleoperated device comprising an interchangeable distal instrument. This device could be designed for use in a surgical environment such that the interchangeable distal instruments would be surgical instruments. However, one of skill in the art will realize that the device could also be deployed in other applications where complex, remote manipulation is required and wherein interchangeable instruments would be useful for different manipulation tasks.

Another aim of the present invention is to provide a mechanical teleoperated system with an improved transmission system. In such a system, the transmission system, and particularly the joints, would be designed such that cables do not rub against each other, thus reducing wear and tear.

### SUMMARY OF THE INVENTION

These aims and other advantages are achieved through a mechanical teleoperated device for remote manipulation designed to naturally replicate the operator's hand movements in the distal area where the manipulation must occur. The mechanical teleoperated device may comprise a slave unit, configured to be driven by a master unit, which is preferably kinematically equivalent to the slave unit. The slave unit has a plurality of slave links interconnected by respective slave joints and comprises a proximal part and a distal part (also referred to as a distal instrument) connected distally to one of the slave joints, referred to as the interface slave joint. More particularly, this interface slave joint of the slave unit may have a coaxial configuration, connecting a proximal and a distal link of the slave unit in such a manner that these two adjoining links are axially rotatable with respect to each other about the axis of the coaxial joint. The master unit comprises a plurality of master links interconnected by respective master joints, and a corresponding interface master joint.

An articulated handle is connected to the master unit for operating the mechanical teleoperated device and an articulated end-effector is connected to the distal extremity of the distal instrument. The mechanical teleoperated device further comprises a transmission system arranged to kinematically connect the equivalent degrees-of-freedom of the master and slave units, including the degrees-of-freedom of the end-effector to the degrees-of-freedom of the handle such that said end-effector replicates the movements of the handle when the mechanical teleoperated device is operated.

The transmission system comprises (i) at least one arrangement of a plurality of rotatable elements coaxially mounted together and coaxial to the axis of the interface slave joint of the slave unit and arranged to rotate independently from each other; (ii) first transmission means integrated into the proximal part of the slave unit and comprising driving cables or the like which are arranged to actuate in rotation each one of the rotatable elements of the said at least one arrangement; and (iii) second transmission means integrated into the distal instrument and comprising driven cables or the like which are arranged to be driven by the rotation of each one of the corresponding rotatable elements of the at least one arrangement of a plurality of rotatable elements.

The transmission system according to the invention is advantageously configured for removably coupling together the first and second transmission means of the respective proximal and distal parts of the slave unit. In this arrangement, each driven cable of said second transmission means is removably connected, by means of additional elements, to one corresponding rotatable element of said at least one arrangement of a plurality of rotatable elements in order to provide a user-friendly interchangeable and modular tooling/instrument system for use with the mechanical teleoperated device.

According to one embodiment of the invention, a segment of each driven cable of the second transmission means is removably connected to one corresponding rotatable element of the at least one arrangement of a plurality of rotatable elements. Each of these segments is guided along a path within a plane which is substantially perpendicular to the axis about which the rotatable elements of the at least one arrangement of a plurality of rotatable elements are able to rotate. This path preferably extends along a circular trajectory, whose center point is coincident with the rotatable elements' axis.

According to another embodiment of the invention, each driven cable of said second transmission means is anchored to a mating receptacle pivotally mounted about the axis of the interface slave joint of the slave unit. Each corresponding rotatable element of the at least one arrangement of a plurality of rotatable elements comprises a complementary mating part removably mounted inside the corresponding mating receptacle.

According to a further embodiment of the invention, each complementary mating part is mounted on a pointer which is connected to one corresponding rotatable element. These pointers are able to rotate around the axis of the interface slave joint and may be angularly offset from each other so that their angular amplitude of movement allows the full-range actuation of the end-effector articulations.

According to a further embodiment of the invention, the mating receptacles and corresponding mating parts are housed inside a slave housing. The slave housing comprises two complementary housing parts removably mounted against each other. Said mating receptacles are mounted inside one complementary housing part which is connected to the distal part of the slave unit whereas said complementary mating parts are mounted inside the other complementary housing part which is connected to the proximal part of the slave unit.

According to another embodiment of the invention, the transmission system comprises two arrangements of a plurality of rotatable elements, wherein rotatable elements of the first arrangement are coaxially mounted together so that each one is able to rotate about the axis of an interface slave joint of the slave unit, and wherein rotatable elements of the second arrangement are coaxially mounted together to rotate about each other, being collinear with the axis of an interface master joint of the master unit. In this configuration, each driving cable of the first transmission means is connected to one rotatable element of the first arrangement of the slave unit and to one corresponding rotatable element of the second arrangement of the master unit such that rotation of each rotatable element of said second arrangement rotates the corresponding rotatable element of said first arrangement.

According to yet another embodiment of the invention, two or more teleoperated devices, as defined above, can be set to work together, enabling the user to perform bi-manual manipulation. In addition, two or more telemanipulators can be mounted on a movable and articulated station, so that they can be easily moved on the ground from one place to the other and their working position and orientation can be tuned in the 3D space.

### BRIEF DESCRIPTION OF THE FIGURES

The invention will be better understood according to the following detailed description of several embodiments of the invention with reference to the attached figures, in which:

FIG. 1 shows a perspective view of a mechanical teleoperated device according to the invention;

FIG. 2 shows a schematic view of the transmission system of the mechanical teleoperated device of FIG. 1;

FIG. 3 shows a schematic view of the transmission system of FIG. 1 comprising first, second and third transmission means;

FIGS. 4, 5 and 6 show a schematic view of the transmission system for the transmission of each degree-of-freedom of the handle to the end-effector;

FIG. 7 shows a perspective view of a detachable distal instrument;

FIG. 8 shows a perspective view of a lower part of the slave housing of the first arrangement of the transmission system containing mating receptacles;

FIG. 9 shows a perspective view of the guiding means of the slave housing of FIG. 8;

FIG. 10 shows an upper part of the slave housing containing complementary mating parts arranged to be removably mounted inside the corresponding mating receptacle of FIG. 8;

FIGS. 11a, 11b and 11c show three different views of the distal part coupling with the proximal part of the slave unit of the mechanical teleoperated device of FIG. 1, and

FIG. 12 shows an exemplary embodiment comprising two identical mechanical teleoperated devices of FIG. 1 mounted on an articulated and movable station, namely a mechanical telemanipulator for surgical procedures.

### DETAILED DESCRIPTION OF THE INVENTION

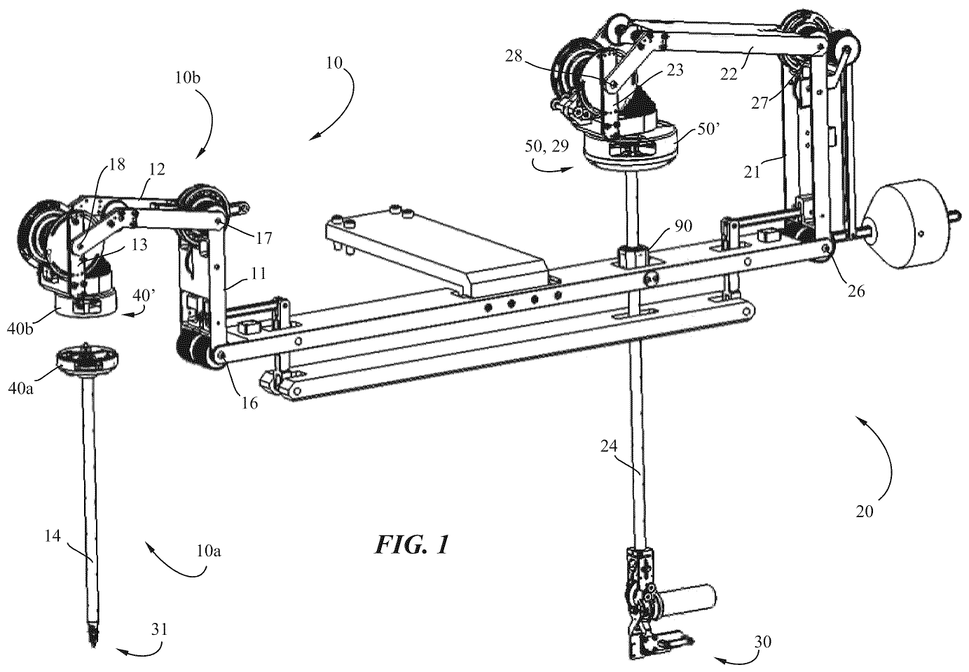

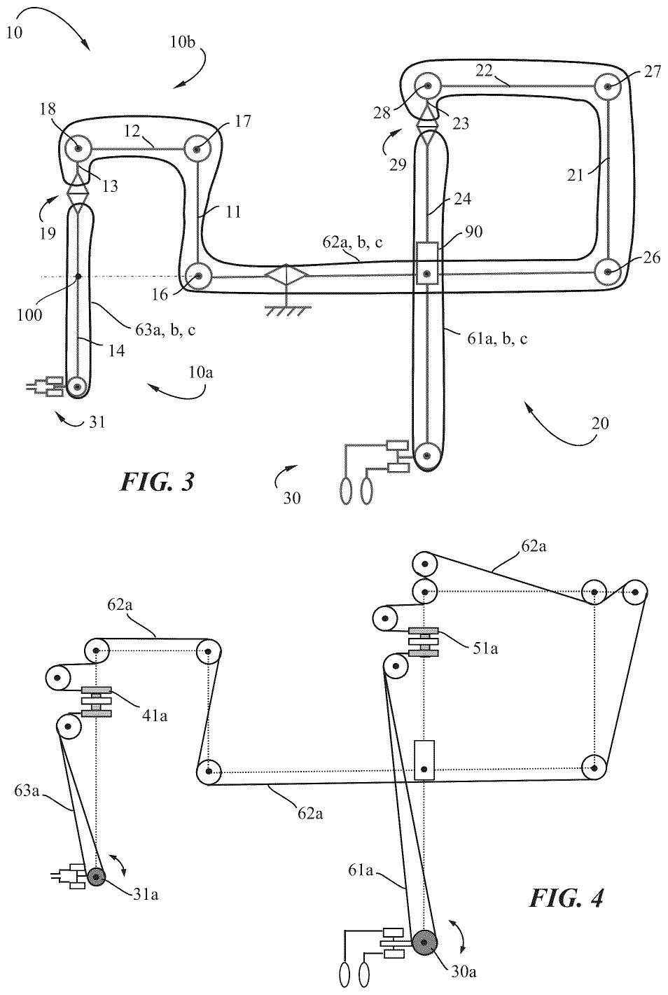

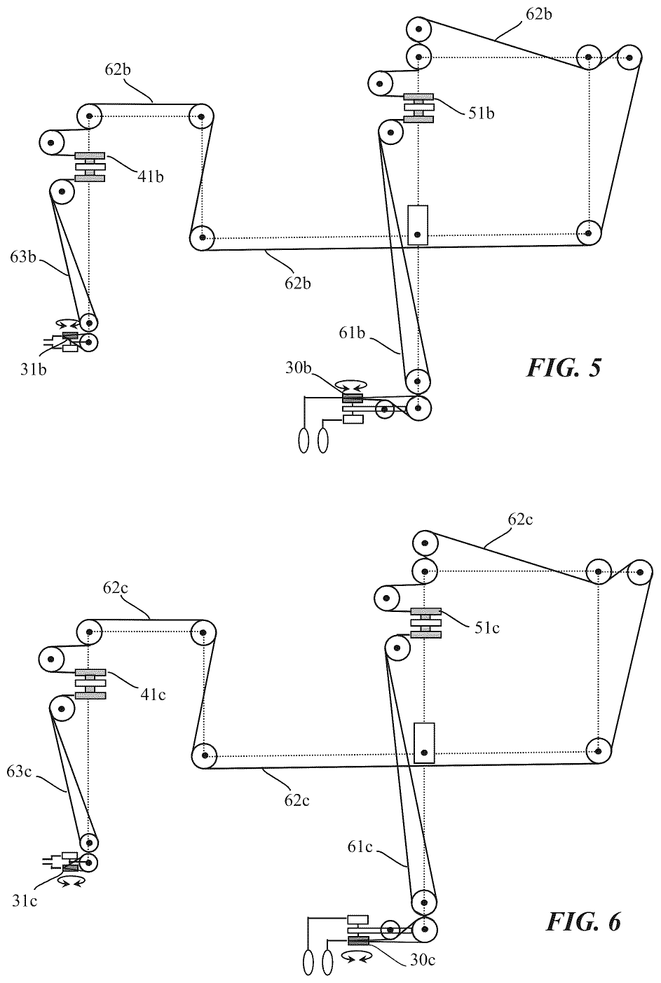

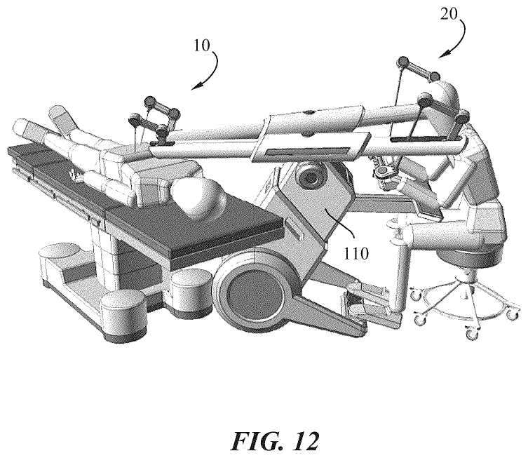

A mechanical telemanipulator, which may be used in minimally invasive surgical procedures or in other applications, constructed in accordance with an embodiment of the present invention, is described herein, and is seen generally in FIG. 12. This manipulator comprises preferably two identical mechanical teleoperated devices, as shown for example in FIG. 1, mounted on an articulated and movable station 110 (FIG. 12) and configured to be operated independently from the other. Each mechanical teleoperated device of FIG. 1, as conceptually described in WO2013/014621, comprises, on the one hand, a master-slave configuration, having a slave unit 10 driven by a kinematically equivalent master unit 20 and, on the other hand, mechanical constraint means 90 such that each part of the slave unit 10 mimics the movement of each corresponding part of the master unit 20 without deviating, during operation of the device, from a remote-center-of-motion (RCM) as shown in FIG. 3. However, in other embodiments of the current invention, the mechanical teleoperated device may not have a remote-center-of-motion (RCM). Given that the two mechanical teleoperated devices are structurally and functionally identical, the description hereafter will refer to one mechanical teleoperated device only as shown particularly in FIGS. 1 to 6.

Referring more particularly to FIG. 3, the slave unit 10 comprises a plurality of slave links 11, 12, 13, 14 interconnected by respective slave joints 16, 17, 18, 19. An articulated end-effector 31 is mounted at the distal end of the slave link 14 which is rotatably connected to the adjoining link 13 through an coaxial joint 19 such that the two adjoining links 13, 14 are coaxially rotatable with respect to each other about the axis of the axial joint 19. The remaining slave joints 16, 17, 18 of the slave unit 10 are of the type of so-called pivot-joint which connects two adjoining links (e.g. pivot joint 17 connecting link 12 with link 13) in order to be angularly actuable with respect to each other within a plane perpendicular to the axis of rotation of said pivot-joint. The master unit 20 comprises a structural and kinematic configuration identical to the slave unit 10. More particularly, the master unit 20 comprises a corresponding plurality of master links 21, 22, 23, 24 interconnected by respective master joints 26, 27, 28, 29. One of these joints is a coaxial joint 29 while the other joints are pivot-joints 26, 27, 28. An articulated handle 30 is connected at the distal end of a master link 24 of the master unit 20 for operating the mechanical teleoperated device.

Upward and downward movements applied to the handle 30 by the operator when the mechanical teleoperated device is in the neutral position of FIG. 3 translate the master link 24 along its longitudinal axis through the mechanical constraint means 90 whereas frontward and backward movements applied on the handle rotate the master link 24 about an axis parallel to the axis of each pivot-joint. Each one of these movements or a combination thereof generates angular displacement of the master joints 26, 27, 28, 29 of the master unit 20 wherein said angular displacement of each master joint causes, through mechanical transmission, an identical angular displacement of the corresponding slave joint 16, 17, 18, 19 of the slave unit 10. The kinematic connection between each pivot-joint 26, 27, 28 of the master unit 20 with the corresponding pivot joint 16, 17, 18 of the slave unit 10, is achieved by pulley-routed cables which are each arranged to be anchored to two corresponding pulleys rotatably mounted around a shaft whose axis is coincident with the axis the corresponding pivot-joint. Kinematic connections between each pivot-joint 16, 17, 18 of the slave unit 10 with the corresponding pivot-joint 26, 27, 28 of the master unit 20 are described in more details in the aforementioned PCT application (WO2013/014621). In this configuration, the kinematic model of the chain formed by the plurality of slave links 11, 12, 13, 14 and respective slave joints 16, 17, 18, 19 of the slave unit 10 is identical to the kinematic model of the chain formed by the plurality of master links 21, 22, 23, 24 and respective master joints 26, 27, 28, 29 of the mater unit 20.

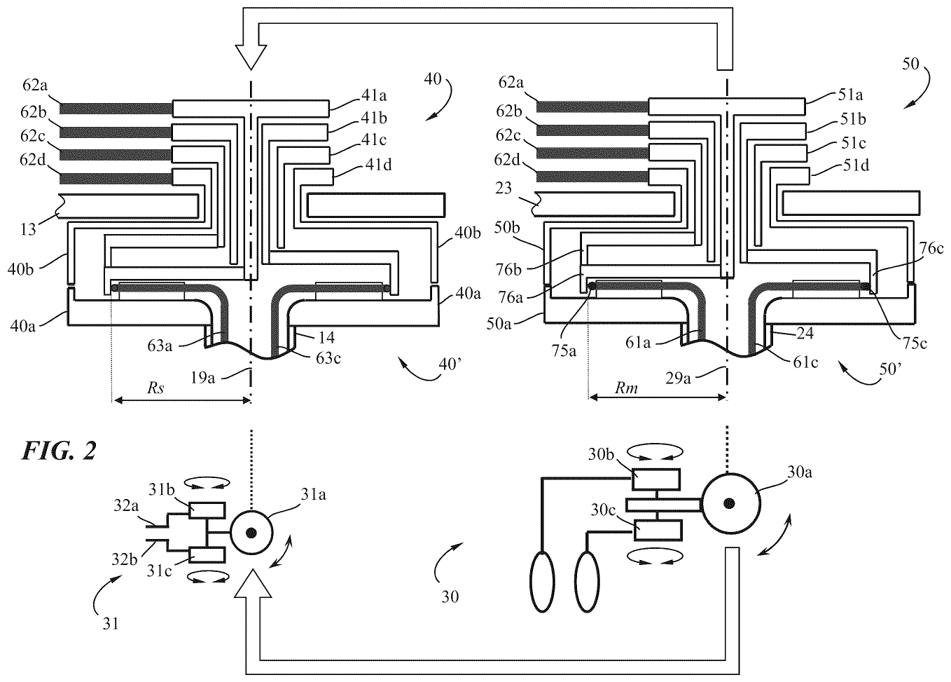

As schematically shown in FIG. 2, the handle 30 is configured to actuate three degrees-of-freedom of the end effector 31 by a transmission system adapted to kinematically connect three pulleys 30a, 30b, 30c of the handle 30 with three corresponding pulley 31a, 31b, 31c of the end-effector 31. In this way, angular displacement of each pulley of the handle 30, when the teleoperated surgical device is operating, is replicated by the corresponding pulley of the end-effector 31. The latter is configured such that counter-rotation of both pulleys 31b, 31c in one direction rotates jaws 32a, 32b in opposite direction away from each other to open the grip of the jaws while counter-rotation of both pulleys 31b, 31c in the other direction rotates the jaws 32a, 32b in opposite directions toward each other to close the grip and maintain a gripping force of the jaws. The working tips of the jaws 32a, 32b have a surface for gripping and may be used, for example, in forceps or cautery applications. Alternately, “gripping” which closes the jaws may be a cutting action when the tips of the jaws 32a, 32b are blades that cooperatively cut as a scissors. Gripping can thus perform different functions depending the nature of the end-effector 31. This end-effector is further configured to pivot about the axis of its pulley 31a when the latter is actuated by the cable-driven system according to the invention.

Still referring to FIG. 2, the transmission system according to this preferred embodiment comprises a first arrangement 40 of three rotatable elements 41a, 41b, 41c coaxially mounted together about the axis 19a of the interface slave joint of the slave unit and a second arrangement 50 of three rotatable elements 51a, 51b, 51c coaxially mounted together about the axis 29a of the interface master joint of the master unit. The rotatable element 41d comprises the driven pulley that actuates the interface slave joint 19 of the slave unit 10, being rigidly attached to the slave link 14. The rotatable element 51d comprises the driving pulley that actuates the interface master joint 29 of the master unit 20, being rigidly attached to the master link 24. Rotatable elements 41a, 41b, 41c of the first arrangement 40 are partly housed inside a slave housing 40′ while rotatable elements 51a, 51b, 51c of the second arrangement 50 are partly housed inside a master housing 50′. As shown in FIG. 1, the slave and master housings 40′, 50′ are connected respectively to a distal link 14 of the slave unit 10 and to a corresponding master link 24 of the master unit 20. The slave housing 40′ is further connected to the rotatable element 41d whereas the master housing 50′ is further connected to the corresponding rotatable element 51d.

The transmission system as shown in FIG. 2 further comprises first transmission means which have three cables 62a, 62b, 62c or similar structure which are each arranged to connect one rotatable element 51a, 51b, 51c of the second arrangement with one corresponding rotatable element 41a, 41b, 41c of first arrangement 40 such that rotation of each rotatable element 51a, 51b, 51c of second arrangement 50 actuates the corresponding cable which rotates in turn the equivalent rotatable element 41a, 41b, 41c of first arrangement 40.

Referring now to FIGS. 4 to 6, the transmission system comprises second transmission means which have three driven cables 63a, 63b, 63c or similar structures which are each arranged to be anchored on a corresponding pulley 31a, 31b, 31c of the end-effector 31 to enable angular displacement of these pulleys about their respective pivotal connection. These driven cables extends axially through the slave link 14 to be removably anchored to a corresponding rotatable element 41a, 41b, 41c of the first arrangement 40 of the slave unit 10. The removable connection of each driven cable to the corresponding rotatable element will be described in details subsequently.

The transmission system further comprises third transmission means which have three cables 61a, 61b, 61c or similar structures which are each arranged to be anchored on a corresponding pulley 30a, 30b, 30c of the handle 30 in order to be driven by angular displacement of these pulleys about their respective pivotal connection generated by the actuation of handle 30. Each of these cables 61a, 61b, 61c extends axially along the master link 24 to be anchored to a corresponding rotatable element 51a, 51b, 51c of the second arrangement 50 of the master unit 20. FIG. 2 partly shows in more details how this type of connection is achieved. Each cable 61a, 61b, 61c (only cables 61a and 61c are visible) are partially guided within a plane which is preferably perpendicular to the axis of the axial joint 29 of the master unit 20 and along a substantially circular path through an angle of less than 120 degrees (on the current embodiment). A segment 75a, 75c (75b is not visible in FIG. 2) of each cables is attached to a corresponding pointer 76a, 76b, 76c, wherein these pointers are coaxially mounted jointly with respective rotatable element 51a, 51b, 51c, and are superposed and angularly offset from each other.

According to the configuration of the transmission system, angular displacement of each pulley 30a, 30b, 30c of the handle 30 about their respective pivotal connection, during operation of the mechanical teleoperated device, drives the corresponding cable 61a, 61b, 61c of the third transmission means which causes the angular displacement of the corresponding rotatable element 51a, 51b, 51c of the second arrangement 50 about the axis of the interface slave joint 29 of the master unit 20. Rotation of each one of these rotatable elements 51a, 51b, 51c drives the corresponding cable 62a, 62b, 62c of the first transmission means which causes the angular displacement of the equivalent rotatable element 41a, 41b, 41c of the first arrangement 40 about the axis of the interface slave joint 19 of the slave unit 10. Rotation of each one of these rotatable elements 41a, 41b, 41c causes in turn the angular displacement of the corresponding pulley 31a, 31b, 31c of the end-effector 31 by means of the corresponding driven cable 63a, 63b, 63c of the second transmission means. The remaining two rotatable elements 41d, 51d of respective slave and master units 10, 20 are connected together by means of cable 62d such that axial rotation of the master link 24 rotates the corresponding slave link 14 about its longitudinal axis.

Due to this particular configuration, the transmission system is advantageously arranged to avoid twisting of the cables, when the master link 24 and the corresponding slave link 14 are coaxially rotating. Wear and tear of the cables resulting from prolonged use of the mechanical teleoperated device are therefore significantly reduced.

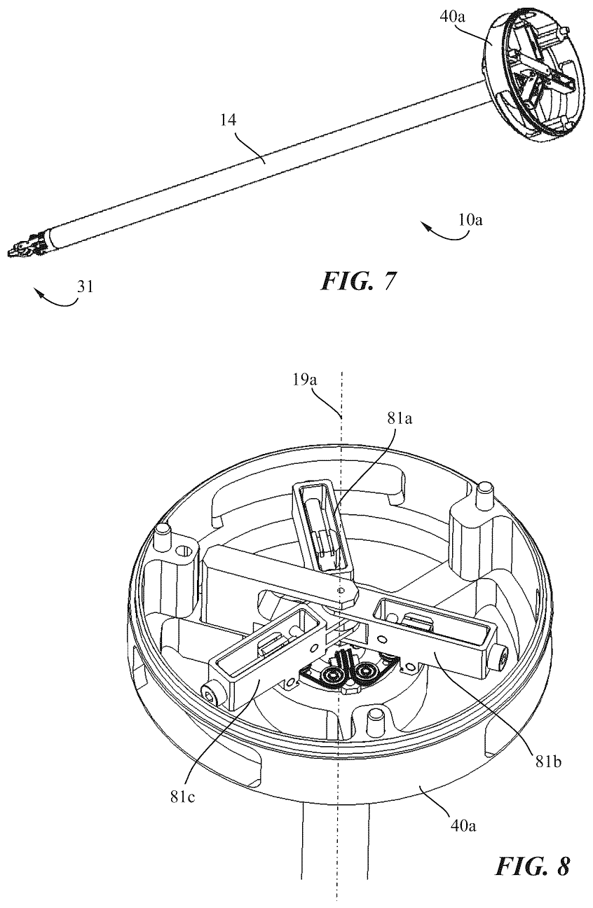

Referring now to FIGS. 8, 9 and 10, the transmission system according to the invention comprises a coupling/decoupling mechanism for removably connecting each driven cable 63a, 63b, 63c of the second transmission means with the corresponding cable 62a, 62b, 62c of the first transmission means in order to provide a user-friendly interchangeable and modular tooling system to the mechanical teleoperated device. To this end, lower and upper parts 40a, 40b of the slave housing 40′ are removably secured against each other preferably by means of screws to allow the user to structurally disconnect the distal part 10a (FIG. 7) from the proximal part 10b of the slave unit 10 as particularly shown in FIG. 1.

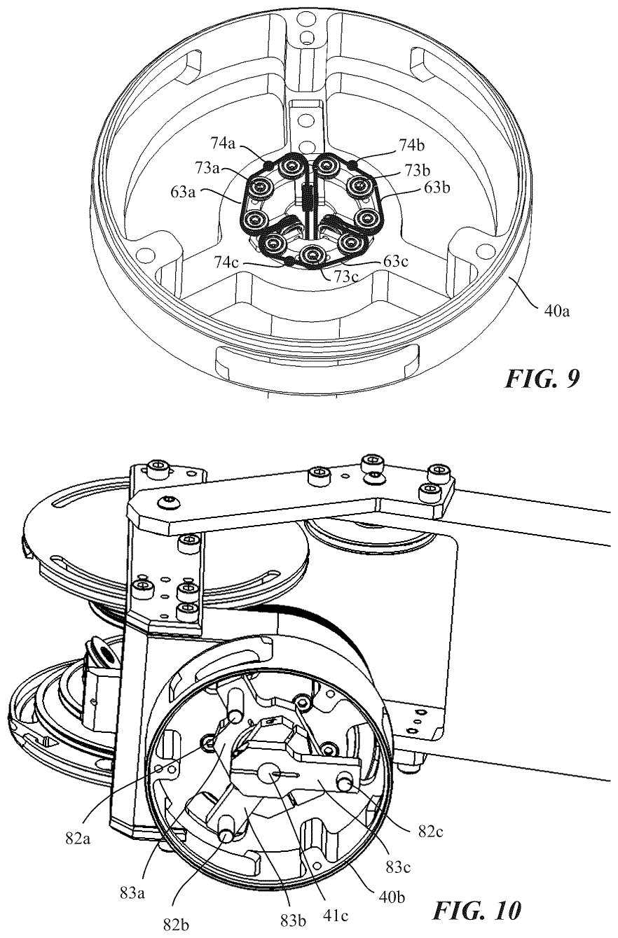

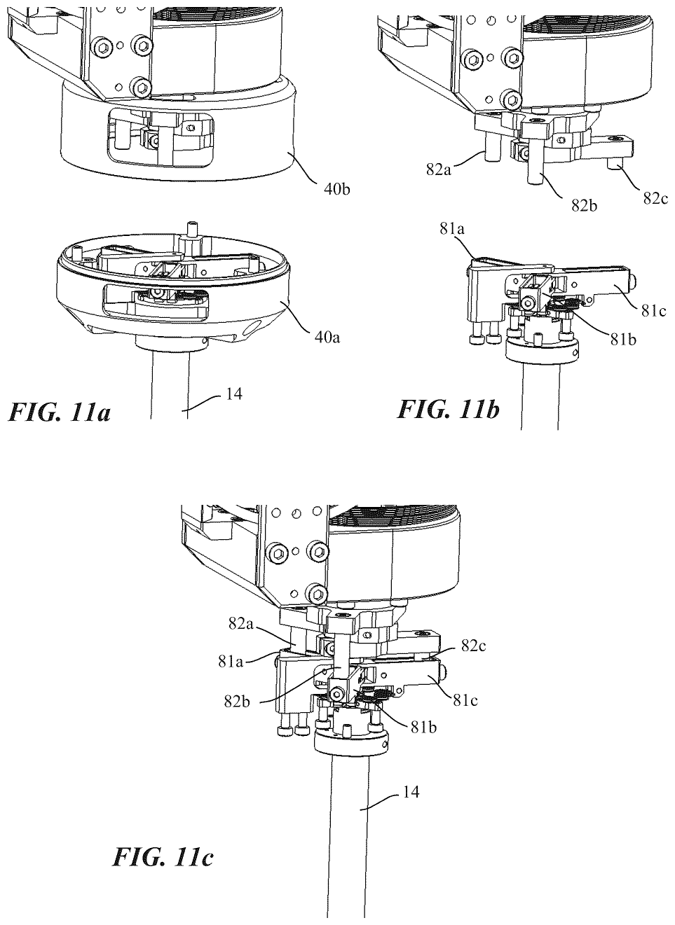

With reference to FIG. 9, the coupling/decoupling device of the transmission system comprises guiding means 73a, 73b, 73c within the lower part 40a of the slave housing. Each of these guiding means 73a, 73b, 73c are preferably in the form of a series of rollers arranged to guide a segment 74a, 74b, 74c of each cable 63a, 63b, 63c of the second transmission means within a plane which is substantially perpendicular to the axis of the interface slave joint 19 of the slave unit 10 and along a substantially circular path through a certain angular range. In other embodiments of the same invention, instead of being composed by a series of rollers, the guiding means may comprise non-moving elements or surfaces in order to guide the segment 74a, 74b, 74c of each cable 63a, 63b, 63c of the second transmission means within their path. Each segment 74a, 74b, 74c of each one of these cables is anchored to a mating receptacle 81a, 81b, 81c pivotally mounted about the axis 19a of said interface slave joint 19 of the slave unit 10 as shown in FIG. 8. As can be seen in FIG. 10, a complementary mating part 82a, 82b, 82c, preferably in the form of a connecting pin, is mounted on respective supports 83a, 83b, 83c which are coaxially arranged jointly with respective rotatable element of the first arrangement. These supports 83a, 83b, 83c which are preferably in the form of pointers are superposed and angularly offset from each other. Each connecting pin 82a, 82b, 82c is adapted to be removably mounted inside the corresponding mating receptacle 81a, 81b, 81c as particularly shown in FIG. 11c.

According to the transmission system, the ratio of Rs/Rm (FIG. 2) equates to the ratio of Ds/Dm wherein:
i) Rs is the radial distance between the axis of the interface slave joint 19 of the slave unit 10 and one anchored segment 73a, 73b, 73c of one driven cable 63a, 63b, 63c of the second transmission means;
ii) Rm is the radial distance between the axis of the interface slave joint 29 of the master unit 20 and one anchored segment of one cable 61a, 61b, 61c of the third transmission means;
iii) Ds is the diameter of one corresponding pulley 31, 31b, 31c of the end-effector 31; and
iv) Dm is the diameter of one corresponding pulley 30a, 30b, 30c of the handle 30.

The configuration of the mechanical teleoperated device according to the invention allows the operator to easily remove the distal part 10a, comprising at its distal end the end-effector 31, and to replace it with another distal part comprising any type of end-effector such as scissors, scalpels, cutters, needle holders or any other surgical accessories.

Although the transmission system as disclosed in this preferred embodiment comprises cables, its particular configuration is not limited to this embodiment. For example, the present invention may also encompasses other flexible and non-flexible drive elements such as rods, linkages or the like which could be arranged to achieve the same functionality set forth herein.

Moreover, although the transmission system of the mechanical teleoperated device according to the preferred embodiment comprises first, second and third transmission means comprising each three cables to actuate three degrees of freedom of the end-effector when the handle is operated, the scope of the invention also covers configurations with first and second transmission means only such that their respective cables circumvents only joint 19 of the slave unit to allow detachment of the distal part 10a from the proximal part 10b of the slave unit 10. Transmission means in excess of three comprising each a distinct set of cables can also be envisaged to cover embodiments in which the master and slave units comprise each several coaxial joints.

While this invention has been particularly shown and described with references to particular embodiments thereof, it will be understood by those skilled in the art that various changes in form and details may be made therein without departing from the spirit and scope of the invention as defined by the appended claims. For example the scope of the invention includes configurations where at least the first and the third transmission means comprise N cables for the transmission of N degrees-of-freedom of the handle to the end-effector, where N can be for example 2, 4 or 5. In addition, the transmission system disclosed according to the embodiments described herein can be adapted to any teleoperated device which does not necessarily comprise a kinematically equivalent master-slave configuration.

1.-15. (canceled)

16. A method for performing a surgery on a patient using a surgical teleoperated device, the method comprising:
moving a handle of the surgical teleoperated device;
responsive to moving the handle, moving a detachable distal instrument of the surgical teleoperated device, the detachable distal instrument comprising an end effector spaced apart from the handle,
responsive to moving the detachable distal instrument, contacting the end effector to tissue of the patient to perform the surgery; and
after the surgery, detaching the detachable distal instrument.

17. The method of claim 16, wherein the detachable distal instrument of the surgical teleoperated device moves responsive to movement of the handle via a transmission system that operatively couples the end effector to the handle.

18. The method of claim 17, wherein detaching the distal instrument comprises removing a plurality of driven elements arranged to cause movement of the end-effector from one or more independently rotatable elements operatively coupled to one or more driving elements of the transmission system.

19. The method of claim 16, further comprising displaying the surgery to permit visualization of the surgery.

20. The method of claim 16, further comprising:
moving a second handle of a second surgical teleoperated device;
responsive to moving the second handle, moving a second detachable distal instrument of the second surgical teleoperated device, the second detachable distal instrument comprising a second end effector spaced apart from the second handle; and
responsive to moving the second detachable distal instrument, contacting the second end effector to tissue of the patient to perform the surgery.
